(19) United States
(12) Patent Application Publication
(10) Pub. No.: US 2011/0288399 A1
(43) Pub. Date: Nov. 24, 2011

(54) PLACENTAL CALCIFICATION MAGNETIC RESONANCE IMAGING

(75) Inventors: Daniel A. Herzka, Rockville, MD (US); Anna T. Fernandez, Croton-on-Hudson, NY (US)

(73) Assignee: KONINKLIJKE PHILIPS ELECTRONICS N.V., EINDHOVEN (NL)

(21) Appl. No.: 13/146,020

(22) PCT Filed: Jan. 12, 2010
(86) PCT No.: PCT/IB10/50103

§ 371 Date: Jul. 25, 2011

Related U.S. Application Data
(60) Provisional application No. 61/151,639, filed on Feb. 11, 2009.

(51) Int. Cl. A61B 5/055

(52) U.S. Cl. 600/410

(57) ABSTRACT

A method comprises acquiring at least one magnetic resonance image of a placenta using an ultra-short echo time (UTE) pulse sequence; and processing the at least one magnetic resonance image to generate information indicative of placental calcification. In apparatus embodiments, a gynecology module (20) comprises a digital processor configured to cause a magnetic resonance imaging scanner (10, 12, 14, 16) to acquire at least one magnetic resonance image of a placenta and to process the at least one magnetic resonance image to generate information indicative of placental calcification.

The following relates to the medical arts, the obstetrics and gynecology arts, the medical imaging arts, and related arts.

In a typical gynecological workflow, a patient is evaluated and classified according to the risk of problems arising during the pregnancy. In accordance with this procedure, some patients are classified as “high risk” meaning that they have a higher than average risk of experiencing problems during the pregnancy. Indicators that may cause a patient to be classified as high risk include patient age, weight, general health, past medical history, or so forth. A patient classified as high risk is typically monitored more closely to ensure that any incipient problems are detected and addressed at an early stage. Toward this end, ultrasound is a usual imaging modality for gynecological monitoring.

Placental calcification has been identified as an indicator or predictor of problematic pregnancies. Calcified placental tissue is more dense than surrounding uncalcified tissue, and accordingly appears as bright features in the ultrasound image. Thus, ultrasound is generally considered to be an effective technique for detecting and monitoring placental calcification.

However, ultrasound has some deficiencies in this regard. Depending upon the placement of the ultrasound probe, the image of the placenta may be partially occluded by the fetus or by other intervening features. As a result, ultrasound may fail to detect occluded calcified placental tissue. Furthermore, placental calcification sometimes forms as “granulations” or small regions of calcification within the larger placental tissue matrix. Because ultrasound is a relatively low resolution imaging technique, it may fail to detect finely granulated calcification. For example, this can be problematic for incipient calcification which is likely to have a finely granulated structure.

The following provides new and improved apparatuses and methods which overcome the above-referenced problems and others.

In accordance with one disclosed aspect, a method comprises: acquiring at least one magnetic resonance image of a placenta; and processing the at least one magnetic resonance image to generate information indicative of placental calcification.

In accordance with another disclosed aspect, a storage medium stores instructions executable by a digital processor to perform a method as set forth in the immediately preceding paragraph.

In accordance with another disclosed aspect, a gynecology module comprises a digital processor configured to cause a magnetic resonance imaging scanner to acquire at least one magnetic resonance image of a placenta and to process the at least one magnetic resonance image to generate information indicative of placental calcification.

One advantage resides in providing placental calcification images of higher resolution than those available via ultrasound.

Another advantage resides in providing unoccluded placental calcification images that are independent of the placement of any medical probe.

Another advantage resides in providing placental calcification images obtained by magnetic resonance imaging.

Yet another advantage resides in providing quantitative analysis of placental calcification images obtained by magnetic resonance imaging.

Further advantages will be apparent to those of ordinary skill in the art upon reading and understand the following detailed description.

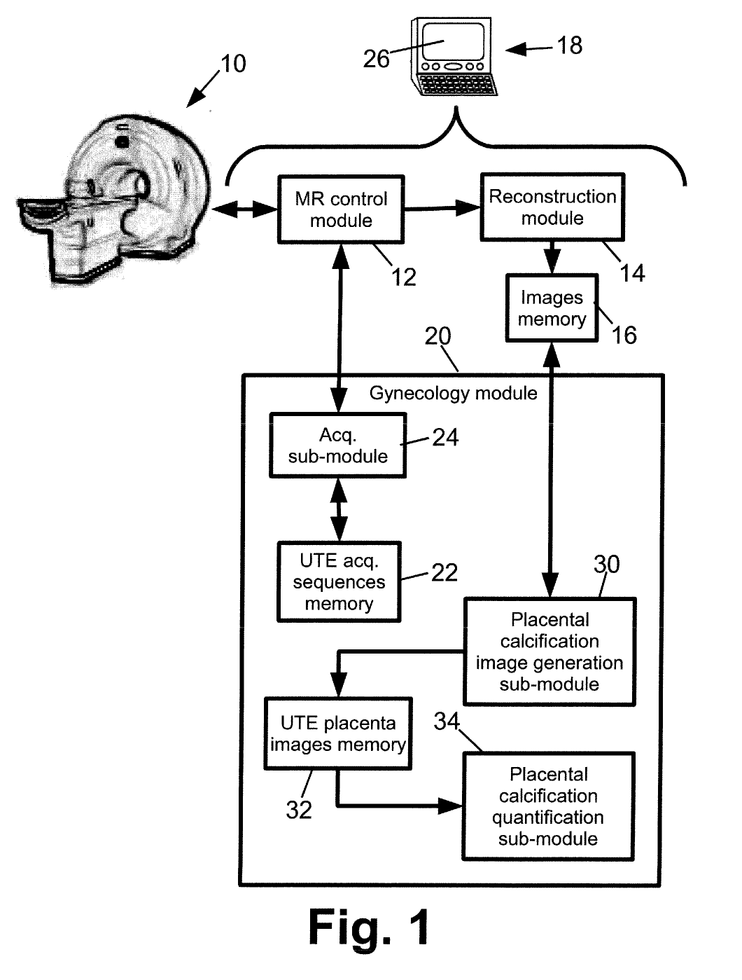

FIG. 1 diagrammatically illustrates a magnetic resonance imaging system configured for gynaecological imaging including placental calcification imaging.

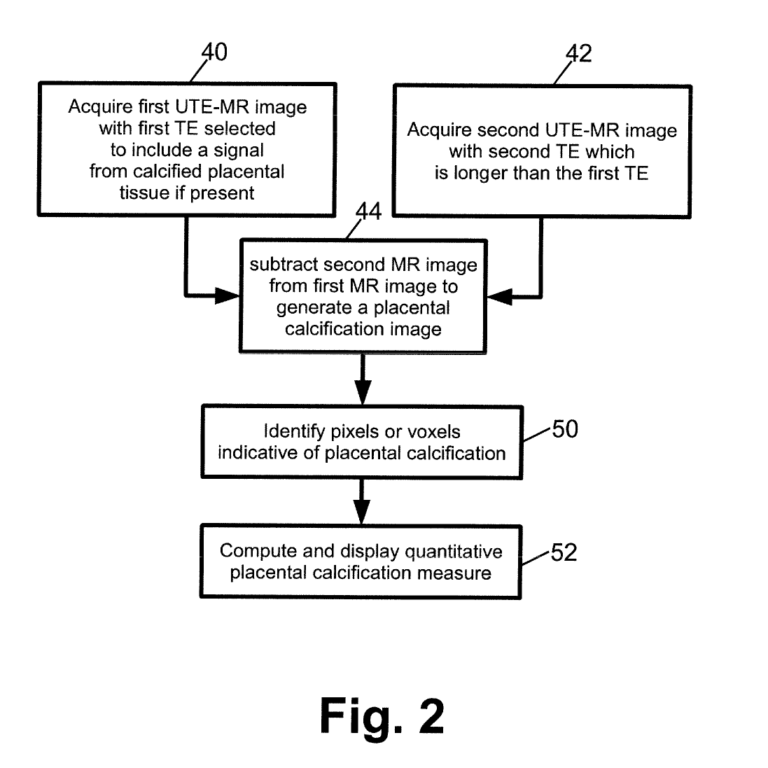

FIG. 2 diagrammatically illustrates a magnetic resonance placental calcification imaging process suitably performed using the system of FIG. 1.

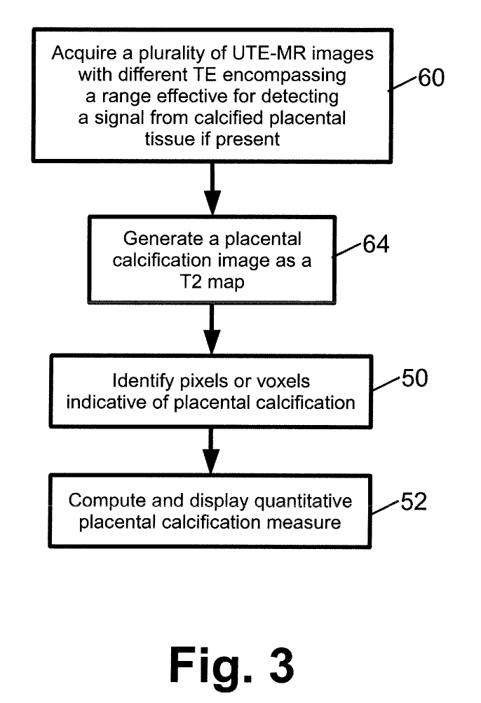

FIG. 3 diagrammatically illustrates a magnetic resonance placental calcification imaging process suitably performed using the system of FIG. 1.

With reference to FIG. 1, a magnetic resonance imaging system configured for gynaecological imaging including placental calcification imaging includes a magnetic resonance imaging scanner 10, such as an illustrated Achieva™ magnetic resonance scanner (available from Koninklijke Philips Electronics N.V., Eindhoven, The Netherlands), or an Intera™ or Panorama™ magnetic resonance scanner (both also available from Koninklijke Philips Electronics N.V.), or another commercially available magnetic resonance scanner, or a non-commercial magnetic resonance scanner, or so forth. In a typical embodiment, the magnetic resonance scanner includes internal components (not illustrated) such as a superconducting or resistive main magnet generating a static (B₀) magnetic field, sets of magnetic field gradient coil windings for superimposing selected magnetic field gradients on the static magnetic field, a radio frequency excitation system for generating a radiofrequency (B₁) field at a frequency selected to excite magnetic resonance (typically ¹H magnetic resonance, although excitation of another magnetic resonance nuclei contained in the placenta is also contemplated), and a radio frequency receive system for detecting magnetic resonance signals emitted from the placenta and surrounding tissue responsive to radiofrequency excitation.

The magnetic resonance imaging scanner 10 is controlled by a magnetic resonance control module 12 to execute a magnetic resonance imaging scan sequence that defines the magnetic resonance excitation, spatial encoding typically generated by magnetic field gradients, and magnetic resonance signal readout. A reconstruction module 14 reconstructs acquired magnetic resonance signals to generate magnetic resonance images that are stored in a magnetic resonance images memory 16. In some embodiments, the components 12, 14, 16 are general-purpose commercial magnetic resonance imaging products provided by the manufacturer of the magnetic resonance imaging scanner 10 and/or by one or more third party vendors, for example embodied as software executing on a digital processor (not shown) of an illustrated computer 18. Alternatively, one or more or all of the components 12, 14, 16 may be custom-built or customer-modified components.

To support gynaecological imaging including placental calcification imaging, a gynaecology module 20 is provided, which may for example be embodied as software executing on a digital processor of the illustrated computer 18. The gynaecology module 20 includes a memory 22 storing one or more ultra-short echo time (UTE) magnetic resonance imaging sequences and an acquisition sub-module 24 that retrieves a selected UTE imaging sequence and causes the selected UTE imaging sequence to be executed by the magnetic resonance imaging scanner 10 under the control of the magnetic resonance control module 12. The UTE imaging sequence employs a short time-to-echo (TE) to enable imaging of calcified placental tissue which typically has a transverse relaxation time (denoted herein as “T2”, sometimes also denoted in the literature as “T₂”) of around 10 microseconds to 2 milliseconds, with the T2 value for given calcified placental tissue depending upon factors such as the extent of calcification.

T2 times of about 10 microseconds to 2 milliseconds which are characteristic of calcified placental tissue generally result in substantially complete decay of the magnetic resonance signal from calcified placental tissue for conventional echo times. Accordingly, in a conventional magnetic resonance imaging sequence acquired using an echo time of order a few milliseconds or longer any calcified placental tissue will appear as voids in the image. Such an image can therefore be interpreted as providing information about calcified placental tissue. However, voids in the image can be due to other sources, such as air pockets, certain vascular structures, or bones, or so forth, and so a void in a conventional magnetic resonance imaging sequence acquired using an echo time of order a few milliseconds or longer cannot be unambigously identified with calcified placental tissue.

In contrast, by using a UTE magnetic resonance acquisition sequence retrieved from the memory 22 with an echo time (also called “time-to-echo”, also denoted by “TE”) that is comparable with or shorter than the T2 relaxation time of calcified placental tissue, the UTE magnetic resonance image shows calcified placental tissue as regions of low, but generally not void, signal intensity. In some embodiments, TE is selected to be less than or about 250 microseconds, which is sufficient to detect calcified placental tissue with T2 relaxation times of order a few tens of microseconds or longer. In some embodiments, TE is selected to be less than or about 100 microseconds, or selected to be less than or about 70 microseconds, which TE values are sufficient to detect calcified placental tissue with T2 relaxation times of order 10 microseconds or longer. In some embodiments, the acquisition sub-module 24 selects or adjusts the echo time of the selected UTE sequence to accommodate a desired placental imaging operation.

Substantially any UTE magnetic resonance imaging sequence can be employed, such as sequences disclosed in Rahmer et al., “Three-Dimensional Radial Ultrashort Echo-Time Imaging with T₂ Adapted Sampling”, Magnetic Resonance in Medicine vol. 55, pages 1075-82 (2006) which is incorporated herein by reference in its entirety or sequences disclosed in Rahmer et al., “Selective 3D ultrashort TE imaging: comparison of ‘dual echo’ acquisition and magnetization preparation for improving short-T₂ contrast”, Magn. Reson. Mater. Phy. (Springer 2007) which is incorporated herein by reference in its entirety. The UTE imaging sequence optionally includes a centric three-dimensional readout in which readout echoes are acquired starting at about a center of k-space. For example, the readout echoes can be acquired using radial readouts initiated at a center of k-space and extending outward from the center of k-space. The acquired magnetic resonance images are preferably volumetric magnetic resonance images that encompass the entire placenta so as to ensure that no regions of calcified placental tissue are missed. The slew rates of the magnetic field gradients are preferably kept below levels that might cause peripheral nerve stimulation (PNS) on the gravida or the fetus, as well as to reduce noise levels within the magnetic resonance imaging scanner 10. A relatively low flip angle and relatively long repeat time (TR) (for example, around 25 milliseconds in some embodiments) advantageously reduces the risk of heating the fetus or the gravida. In some embodiments using these acquisition parameters, a complete volumetric magnetic resonance image of the placenta can be acquired in about 3-5 minutes.

Execution by the scanner 10 and control module 12 of a UTE magnetic resonance sequence retrieved from the memory 22 by the acquisition sub-module 24 with the placenta disposed in a field of view of the magnetic resonance imaging scanner 10 results in UTE magnetic resonance imaging data that are reconstructed by the reconstruction module 14 to produce one or more UTE magnetic resonance images of the placenta. The one or more UTE magnetic resonance images are suitably stored in the images memory 16, displayed on a display 26 of the computer 18, or otherwise stored and/or utilized.

The generated UTE magnetic resonance image or images of the placenta show calcified placental tissue with low intensity, but not as void regions. Accordingly, calcified placental tissue is imaged, but may be obscured by other brighter regions corresponding to normal placental tissue or other tissue with longer T2 relaxation times. Due to the short echo time (for example, about 60-70 microseconds in some embodiments), most magnetic resonance signal degradation mechanisms do not have sufficient time to affect the UTE magnetic resonance image. However, the short T2 relaxation time of calcified placental tissue ensures that calcified placental tissue appears with a low (but generally not void) magnetic resonance signal in the UTE magnetic resonance images. This makes interpretation of the images relatively straightforward.

Although a UTE magnetic resonance image provides relatively unambigous identification of calcified placental tissue, the clinical value of such an image, or a set of two or more such images, is optionally enhanced by post-reconstruction image processing. Toward this end, in the illustrated embodiment a plurality of (that is, two or more) UTE magnetic resonance images acquired with two or more different echo times are optionally processed by a placental calcification image generation sub-module 30 of the gynaecology module 20 in order to generate a placental calcification image that is stored in a memory 32, displayed on the display 26 of the illustrated computer 18 or another display device, or otherwise utilized. Optionally, the placental calcification image is further processed by a placental calcification quantification sub-module 34 to define a placental calcification measure, and a perceivable representation of the placental calcification measure is output. For example, the perceivable representation of the placental calcification measure can be a numerical value displayed on the display 26 of the computer 18 or another display device, or the placental calcification measure can be represented by the length of a displayed bar or other graphical representation.

With reference to FIG. 2, a flowchart illustrates a magnetic resonance placental calcification imaging process that may be suitably performed by the gynaecology module 20. In a first acquisition operation 40, a first UTE magnetic resonance image is acquired with a first echo time (TE) selected to include a signal from calcified placental tissue if present in the placenta undergoing imaging. The first echo time should be short enough that the magnetic resonance signal from any calcified placental tissue has not decayed to substantially zero due to the short T2 before the magnetic resonance readout. In a second acquisition operation 42, a second UTE magnetic resonance image is acquired with a second echo time (TE) that is longer than the first echo time. The second echo time may optionally be sufficiently long to ensure the magnetic resonance signal from any calcified placental tissue has decayed to substantially zero, so that any calcified placental tissue appears as void regions in the second UTE magnetic resonance image. Indeed, although FIG. 2 illustrates the second acquisition operation 42 as a UTE imaging operation which has advantages in terms of acquisition speed, it is contemplated for the second acquisition operation 42 to be a conventional acquisition that does not use an ultra-short echo time. Alternatively, the second echo time may optionally be longer than the first echo time but still short enough that the magnetic resonance signal from any calcified placental tissue has not yet decayed to substantially zero, so that any calcified placental tissue appears as low intensity, but not void, regions in the second magnetic resonance image.

With continuing reference to FIG. 2, the placental calcification image generation module 30 suitably performs a subtraction operation 44 in which the second magnetic resonance image is subtracted from the first magnetic resonance image to generate a placental calcification image. The effect of subtraction is to suppress the magnetic resonance signal from non-calcified tissue for which pixel intensities in the first and second images should be about the same, so that any regions of calcified placental tissue appear as brighter regions due to the larger difference in calcified placental tissue intensity in the first and second images. This resulting image can be displayed on the display 26 of the computer 18 or on another display device. In an identification operation 50, pixels or voxels of the placental calcification image that are indicative of placental calcification are identified. This identification can be done, for example, using intensity thresholding. A quantification operation 52 then suitably computes a quantitative placental calcification measure, for example by counting the number of identified placental calcification pixels or voxels. The count is optionally normalized by the total count of pixels or voxels in the image, or of the placenta, to produce a quantitative placental calcification measure as a fractional volume or per-unit volume measure. The quantitative placental calcification measure is suitably displayed as a quantitative numerical value or quantitative graphical representation on the display 26 of the illustrated computer 18 or on another display device.

In some embodiments, the quantification operation 52 outputs the quantitative placental calcification measure as a single quantitative value that assesses the extent of placental calcification indicated by the placental calcification image. Gynaecologists, clinicians, or other medical personnel can refer to this single quantitative value as a quantitative result of the magnetic resonance-based placental calcification “test” and can use the quantitative value to draw boundaries for selection of one treatment as compared with another treatment. As an illustrative example, the quantification operation 52 is contemplated to return a single quantitative value termed the “total volume of calcified placenta” or “Calcified Placenta Percentage” computed as a ratio of the calcified volume (suitably represented, for example, by the count of placental calcification pixels or voxels identified by the operation 50) and the total placental volume (suitably represented, for example, by the total number of pixels representing the placenta, suitably delineated by a manual or automated thresholding operation), optionally multiplied by a factor of 100 in order to convert the value to a percentage.

With reference to FIG. 3, a flowchart illustrates another magnetic resonance placental calcification imaging process that may be suitably performed by the gynaecology module 20. In an acquisition operation 60, a plurality of UTE magnetic resonance images are acquired with different echo times encompassing a range effective for detecting a signal from calcified placental tissue, if present in the placenta undergoing imaging. In other words, the range of different echo times should include echo times that are sufficiently short so that the magnetic resonance signal from any calcified placental tissue will not have decayed substantially to zero before the magnetic resonance signal readout phase of the UTE imaging sequence. The placental calcification image generation module 30 then suitably performs a T2 mapping operation 64 in which signal intensity-versus-TE relationships are generated on a per-pixel or per-voxel basis from the images acquired in the operation 60, and T2 values are mapped based on a slope or other T2-indicative characteristic of the signal intensity-versus-TE relationships. In this approach, regions of calcified placental tissue are expected to be revealed by their typically short T2 decay times of around 10 microseconds to 2 milliseconds. The generated T2 map thus serves as the placental calcification image, and may be thresholded by the placental calcification pixel or voxel identification operation 50 and further processed by the quantification operation 52 as already described with reference to FIG. 2.

The resulting information, for example in the form of a placental calcification image and/or a quantitative placental calcification measure, is suitably considered by a gynaecologist or other appropriate medical specialist in assessing the condition of the patient and the state of the pregnancy. For example, identification of a certain density or amount of calcified placental tissue may be taken as an indication of a certain pregnancy complication. Advantageously, since the placental calcification image derived from the UTE magnetic resonance image or images is three-dimensional and generally does not suffer from occlusion due to neighboring features, the gynaecologist or other appropriate medical specialist can use the placental calcification image to detect regions of placental calcification substantially anywhere within the placenta. Accordingly, clinical significance may optionally be accorded to the location within the placenta of any such regions of placental calcification. Still further, detection of relatively finely granulated regions of placental calcification is facilitated by the relatively high resolution of the UTE magnetic resonance images.

Referring back to FIG. 1, it is to be understood that the gynecology module 20 and its constituent processing sub-modules 30, 34 can be suitably embodied by substantially any digital processor or combination of digital processors that is programmed to define a suitable method such as those illustrated in FIGS. 2 and 3. In some embodiments, the acquisition sub-module 24 and associated UTE sequence or sequences 22 may be embodied by a first digital processor and optional ancillary components such as memory devices, application specific integrated circuitry (ASIC) for performing selected computationally intensive operations, or so forth; while, the post-reconstruction processing components 30, 34 are embodied by a second digital processor that may be different from, or the same as, the first digital processor.

Furthermore, the disclosed processing may be embodied as a storage medium storing instructions that are executable by a digital processor to perform the disclosed image acquisition and/or processing operations. Such a storage medium may, for example, include one or more of the following: a magnetic storage medium such as a hard drive (not shown) of the computer 18; an optical memory such as an optical disk and corresponding optical disk drive (not shown) of the computer 18; a FLASH or other electrostatic memory; a random access memory (RAM); a read-only memory (ROM); or so forth. In some embodiments, the storage medium may be associated with a server logically disposed on and accessed via a hospital (or other local area) network or the Internet.

The invention has been described with reference to the preferred embodiments. Modifications and alterations may occur to others upon reading and understanding the preceding detailed description. It is intended that the invention be construed as including all such modifications and alterations insofar as they come within the scope of the appended claims or the equivalents thereof.

1. A method comprising:
acquiring at least one magnetic resonance image of a placenta; and
processing the at least one magnetic resonance image to generate information indicative of placental calcification.

2. The method as set forth in claim 1, wherein the acquiring comprises:
acquiring at least one ultra-short echo time (UTE) magnetic resonance image.

3. The method as set forth in claim 1, wherein the acquiring comprises:
acquiring at least one magnetic resonance image using an echo time of less than or about 250 microseconds.

4. The method as set forth in claim 1, wherein the acquiring comprises:
acquiring at least one magnetic resonance image using an echo time of less than or about 100 microseconds.

5. The method as set forth in claim 1, wherein the acquiring comprises:
acquiring at least one magnetic resonance image using an echo time of less than or about 70 microseconds.

6. The method as set forth in claim 1, wherein the acquiring comprises:
acquiring at least one magnetic resonance image using a centric three-dimensional readout in which readout echoes are acquired starting at about a center of k-space.

7. The method as set forth in claim 1, wherein the acquiring comprises:
acquiring at least one magnetic resonance image using radial readouts initiated at a center of k-space and extending outward from the center of k-space.

8. The method as set forth in claim 1, wherein the acquiring comprises:
acquiring at least one volumetric magnetic resonance image.

9. The method as set forth in claim 1, wherein the acquiring comprises:
acquiring a first magnetic resonance image with a first echo time selected such that the first magnetic resonance image will include a signal from calcified placental tissue if calcified placental tissue is present in the placenta; and
acquiring a second magnetic resonance image with a second echo time longer than the first echo time.

10. The method as set forth in claim 9, wherein the second echo time is selected such that the second magnetic resonance image will not include a signal from calcified placental tissue even if calcified placental tissue is present in the placenta.

11. The method as set forth in claim 9, wherein the processing comprises:
subtracting the second magnetic resonance image from the first magnetic resonance image to generate a placental calcification image.

12. The method as set forth in claim 9, wherein the processing comprises:
combining the second magnetic resonance image and the first magnetic resonance image to generate a placental calcification image.

13. The method as set forth in any one of claims 12, wherein the processing further comprises:
identifying pixels or voxels of the placental calcification image corresponding to placental calcification; and
computing a quantitative measure of placental calcification based on the identified pixels or voxels.

14. The method as set forth in claim 1, wherein the acquiring comprises acquiring a plurality of magnetic resonance images with different echo times, and the processing comprises:
generating a T2 map based on the plurality of magnetic resonance images.

15. The method as set forth in claim 14, wherein the acquiring a plurality of magnetic resonance images with different echo times encompasses echo times effective for the T2 map to have a T2 resolution encompassing a T2 time or range of T2 times of calcified tissue of the placenta.

16. A storage medium storing instructions executable by a digital processor to perform a method as set forth in claim 1.

17. A gynecology module comprising a digital processor configured to cause a magnetic resonance imaging scanner to acquire at least one magnetic resonance image of a placenta and to process the at least one magnetic resonance image to generate information indicative of placental calcification.

18. The gynecology module as set forth in claim 17, comprising:
a first sub-module comprising a first digital processor configured to cause a magnetic resonance imaging scanner to acquire at least one ultrashort time-to-echo (UTE) magnetic resonance image of a placenta; and
a second sub-module comprising a second digital processor that is the same as or different from the first digital processor, the second digital processor configured to process the at least one UTE magnetic resonance image to generate information indicative of placental calcification.

19. The gynecology module as set forth in claim 18, wherein:
the first sub-module is configured to cause a magnetic resonance imaging scanner to acquire a plurality of UTE magnetic resonance images of a placenta with different echo times; and
the second sub-module is configured to process the plurality of UTE magnetic resonance images to generate a placental calcification image.

20. The gynecology module as set forth in claim 19, wherein the second sub-module is further configured to (i) identify and quantify pixels or voxels of the placental calcification image corresponding to placental calcification to define a placental calcification measure and (ii) output a perceivable representation of the placental calcification measure.

21. The gynecology module as set forth in claim 19, further comprising:
a display device configured to display the placental calcification image.
